(19) United States
(12) Patent Application Publication
(10) Pub. No.: US 2022/0295791 A1
(43) Pub. Date: Sep. 22, 2022

(54) METHODS FOR VIRAL INACTIVATION BY ENVIRONMENTALLY COMPATIBLE DETERGENTS

(71) Applicant: Eli Lilly and Company, Indianapolis, IN (US)

(72) Inventors: Wen Luo, Carmel, IN (US); Sean Michael O'Donnell, Zionsville, IN (US)

(21) Appl. No.: 17/830,726

(22) Filed: Jun. 2, 2022

Related U.S. Application Data
(63) Continuation of application No. PCT/US2020/063531, filed on Dec. 7, 2020.
(60) Provisional application No. 62/947,276, filed on Dec. 12, 2019.

(51) Int. Cl. A01N 43/16; A01N 25/22; A01N 25/04; A01P 1/00

(52) U.S. Cl. CPC A01N 43/16; A01N 25/22; A01N 25/04; A01P 1/00

(57) ABSTRACT

The invention provides methods of using environmentally compatible detergents in the field of recombinant protein manufacturing for inactivating viruses in a product feedstream in the manufacturing process of proteins intended for administration to a patient, such as therapeutic or diagnostic proteins. The invention further provides methods wherein the environmentally compatible detergent used in the present invention maintains product quality of the therapeutic or diagnostic protein whilst effectively inactivating viruses.

The present invention relates to the field of recombinant protein manufacturing. More particularly, the present invention provides a method for inactivating viruses in a feedstream in the manufacturing process of proteins intended for administration to a patient, such as therapeutic or diagnostic proteins. The present invention further provides a method wherein the environmentally compatible detergent of the invention effectively provides anti-viral activity and maintains product quality of the therapeutic or diagnostic protein. The methods provide important real-world biological product manufacturing advantages, for example, as compared to methods using Triton X-100, and/or methods which do not use a detergent.

Detergents are key reagents in the manufacturing process of therapeutic or diagnostic proteins to ensure safety of the final biological product. Viral contamination in the final biological product can have serious clinical consequences arising from, for example, contamination of the source cell lines themselves or from adventitious introduction of viruses during the various manufacturing steps. Thus, detergents are used for purposes of removing viral contaminants that are present in the feedstream during the manufacturing process of the therapeutic or diagnostic protein. After purification of the desired protein, the growth media and buffers used in the product manufacturing and purification process are inactivated and discharged to the wastewater. However, such discharge which contains the detergents used in the manufacturing and purification process may raise environmental concerns as is estimated by environmental risk assessment (ERA) for detergents.

Current methods for inactivating viruses in the manufacturing process of bioproducts such as therapeutic or diagnostic proteins rely heavily on the use of the detergent Triton X-100. Triton X-100 is a polyoxyethylene ether which typically achieves robust enveloped viral inactivation, greater than 4 logs, under a diverse set of experimental conditions. However, 4-(1,1,3,3-tetramethylbutyl) phenol (aka 4-tert-octylphenol), a degradation product of Triton X-100, has been identified as a potential environmental toxin to wildlife, including possible estrogenic effects. Thus, removal of 4-tert-octylphenol from waste streams is desired but would require complex, lengthy and costly measures. Thus, due to the toxicity concerns, the EU-REACH committee voted to add Triton X-100 to Annex XIV, a list of banned substances in the EU, prohibiting the use of Triton X-100 in recombinant protein manufacturing after 2021. Alternative agents to replace Triton X-100 in the manufacture of biologics are not well established. Thus, there is an urgent, time sensitive need for methods of effectively inactivating viruses during the manufacturing processes of biologics such as therapeutic or diagnostic proteins that are environmentally less toxic than methods incorporating Triton X-100.

Environmentally compatible alternatives to Triton X-100 for inactivating viruses, particularly enveloped viruses, in the manufacturing process of a protein have been explored such as in WO 2019/121846 and in WO 2015/073633. However, the effects of using different detergents for viral inactivation are variable. Some detergents are not suitable for use in the manufacturing process due to insolubility or are difficult to remove from the feedstream. Further, some detergents increase the turbidity and/or foaming of the feedstream, thus requiring added measures to decrease the turbidity and/or foaming of the feedstream. Other detergents are not as effective at broad temperature and pH ranges and require higher concentrations for viral inactivation. The combination of these variations can result in added cost and time to the manufacturing process of the protein. In addition, the effectiveness of many detergents in the manufacturing feedstream of different types and sizes of proteins are not sufficient.

Thus, there remains a need for methods of effectively inactivating viruses during the manufacture of therapeutic or diagnostic proteins of different types and sizes, using an environmentally compatible detergent that effectively inactivates different enveloped viruses at both low and high temperature and pH ranges, in a reasonable amount of time and at reasonable concentrations of such detergent(s), and/or is aqueous, soluble and/or is readily solubilized, and/or does not affect turbidity of the feedstream so that it is amenable to large scale manufacturing. Further, it is preferred that the detergent is sufficiently removed from the product feedstream (to meet regulatory requirements) at the purification step, without added complex, lengthy, and/or costly measures, and that the product quality of the therapeutic or diagnostic protein is maintained.

Accordingly, the present invention addresses one or more of the above problems by providing methods of inactivating viruses in the manufacturing process of therapeutic or diagnostic proteins. Surprisingly, the methods of the present invention provide environmentally compatible detergents that are highly effective at inactivating viruses at broad temperature, and/or pH ranges, in the manufacture of different types of proteins, in a reasonable amount of time, and at a reasonable concentration range without toxic environmental impact. Surprisingly, the methods of the present invention further provide use of environmentally compatible detergents that readily achieve complete viral inactivation, that is at least as comparable to Triton-X 100, and where the environmentally compatible detergents have no known environmental toxicities at the concentrations used. Further, the methods of the present invention achieve complete viral inactivation, with a viral log reduction factor (“LRF”) of greater than about 3 to greater than about 6 at temperature range of about 4° C. to at least about 30° C. and at a broad pH range, in the manufacture of different types and sizes of therapeutic or diagnostic proteins in a reasonable amount of time, and at a reasonable concentration range without environmental impact. Surprisingly, methods of the present invention also achieved viral inactivation in all enveloped viruses tested at a temperature range of about 4° C. to at least about 30° C. within greater than 1 to about 180 minutes, at a broad pH range, in the manufacture of different types and sizes of therapeutic or diagnostic proteins at a reasonable concentration range.

Accordingly, the present invention provides a method of inactivating viruses in a feedstream comprising adding to the feedstream detergents that are environmentally compatible. The exemplary detergents used in the methods of the present invention are aqueous solutions, that do not significantly affect the turbidity of the feedstream and, thus, are amenable to large scale manufacturing of therapeutic or diagnostic proteins. Further, the detergents used in the present invention are sufficiently and effectively removed (to meet regulatory requirements) at the purification step with or without a prior filtration step. Furthermore, no complex, lengthy and/or costly measures are required to remove the detergents from the feedstream. Additionally, the exemplary detergents used in the methods of the present invention do not negatively impact the final quality of the therapeutic or diagnostic protein. The detergents used in the methods of the present invention effectively inactivate enveloped viruses at broad temperature and pH ranges, without toxic environmental impact.

In accordance with one aspect of the invention, a method for inactivating viruses with an alkyl glycoside detergent in a feedstream in the manufacturing process of therapeutic or diagnostic proteins is provided. In accordance with another aspect of the invention, the detergent used in the methods of the present invention comprises greater than about 40% undecyl alkyl glycoside. In another aspect of the invention, the detergent used in the present invention comprises greater than about 40% undecyl alkyl glycoside and less than about 20% other alkyl glycosides. In another aspect of the invention, the detergent used in the present invention comprises greater than about 40% undecyl alkyl glycoside and less than about 10% decyl alkyl glycoside. In yet a further embodiment, the detergent used in the methods of the present invention comprises about 40% to about 60% undecyl alkyl glycoside. In yet a further embodiment, the detergent used in the methods of the present invention comprises about 53% to about 57% undecyl alkyl glycoside. In another aspect of the invention, the detergent used in the present invention comprises greater than about 50% undecyl alkyl glycoside. In yet a further embodiment, the detergent used in the methods of the present invention comprises about 40% to about 60% undecyl alkyl glycoside and less than about 20% other alkyl glycosides. In yet a further embodiment, the detergent used in the methods of the present invention comprises about 40% to about 60% undecyl alkyl glycoside and less than about 10% decyl alkyl glycoside. In yet a further embodiment, the detergent used in the methods of the present invention comprises about 53% to about 57% undecyl alkyl glycoside and less than about 20% other alkyl glycosides. In yet a further embodiment, the detergent used in the methods of the present invention comprises about 53% to about 57% undecyl alkyl glycoside and less than about 10% decyl alkyl glycoside. In yet further embodiments the detergent used in the methods of the present invention comprises zero or less than about 0.1% 0.5%, 1%, 2%, 3%, 4%, 5%, 6%, 7%, 8%, 9%, 10%, 11%, 12%, 13%, 14%, 15%, 16%, 17%, 18%, 19% or 20% of other alkyl glycosides. In some embodiments of the invention the other alkyl glycosides comprise of a mixture of nonyl alkyl glycoside, decyl alkyl glycoside and/or lauryl alkyl glycoside. In yet further embodiments the detergent used in the methods of the present invention comprises zero or less than about 0.1% 0.5%, 1%, 2%, 3%, 4%, 5%, 6%, 7%, 8%, 9%, 10% of decyl alkyl glycoside. In one embodiment of the invention, the detergent used in the methods of the present invention comprise an undecyl alkyl glycoside.

In one embodiment of the invention, the detergent used in the methods of the present invention has a CAS registry number of CAS 98283-67-1. In yet other embodiments, the detergent used in the methods of the present invention is SIMULSOL™ SL 11W. In a further embodiment, the SIMULSOL™ SL 11W used in the methods of the present invention comprises greater than about 40% undecyl alkyl glycoside. In yet a further embodiment, the SIMULSOL™ SL 11W used in the methods of the present invention comprises about 40% to about 60% undecyl alkyl glycoside. In yet a further embodiment, the SIMULSOL™ SL 11W used in the methods of the present invention comprises about 53% to about 57% undecyl alkyl glycoside. In yet a further embodiment, the SIMULSOL™ SL 11W used in the methods of the present invention comprises about 40% to about 60% undecyl alkyl glycoside and less than about 10% decyl alkyl glycoside. In yet a further embodiment, the SIMULSOL™ SL 11W used in the methods of the present invention comprises about 53% to about 57% undecyl alkyl glycoside and less than about 10% decyl alkyl glycoside. In yet further embodiments the SIMULSOL™ SL 11W used in the methods of the present invention comprises zero or less than about 0.1% 0.5%, 1%, 2%, 3%, 4%, 5%, 6%, 7%, 8%, 9%, 10%, 11%, 12%, 13%, 14%, 15%, 16%, 17%, 18%, 19% or 20% of other alkyl glycosides. In embodiments of the invention the other alkyl glycosides in the SIMULSOL™ SL 11W used in the methods of the present invention comprises a mixture of nonyl alkyl glycoside, decyl alkyl glycoside and/or lauryl alkyl glycoside. In yet further embodiments the SIMULSOL™ SL 11W used in the methods of the present invention comprises zero or less than about 0.1% 0.5%, 1%, 2%, 3%, 4%, 5%, 6%, 7%, 8%, 9%, 10% of decyl alkyl glycoside. In one embodiment of the invention, the SIMULSOL™ SL 11W used in the methods of the present invention is an undecyl alkyl glycoside.

In yet another embodiment, the detergent used in the methods of the present invention is SIMULSOL™ SL 82. In yet a further embodiment, the SIMULSOL™ SL 82 used in the methods of the present invention comprises about 40% to about 60% undecyl alkyl glycoside.

In a further embodiment, the invention provides methods of inactivating viruses in a feedstream comprising, adding to the feedstream an environmentally compatible detergent at a final concentration of about 0.05% w/w to about 1% w/w, wherein the environmentally compatible detergent comprises greater than about 40% decyl alkyl glycoside. In some embodiments, the detergent used in the methods of the present invention is added to the feedstream to a final concentration of about 0.1% w/w to about 1% w/w. In some embodiments, the detergent used in the methods of the present invention is added to the feedstream to a final concentration of 0.1% w/w to 1% w/w. In some embodiments, the detergent is added to the feedstream to a final concentration of about 0.3% w/w to about 1% w/w. In some embodiments, the detergent used in the methods of the present invention is added to the feedstream to a final concentration of 0.3% w/w to 1% w/w. In some embodiments, the detergent used in the methods of the present invention is added to the feedstream to a final concentration of about 0.1% w/w to about 0.3% w/w. In some embodiments, the detergent used in the methods of the present invention is added to the feedstream to a final concentration of 0.1% w/w to 0.3% w/w. In yet further embodiments, the detergent used in the methods of the present invention is added to the feedstream to a final concentration of about 0.3% w/w. In yet further embodiments, the detergent used in the methods of the present invention is added to the feedstream to a final concentration of 0.3%. In yet further embodiments the detergent used in the methods of the present invention is added to the feedstream to a final w/w concentration of about 0.01%, about 0.05%, about 0.1%, about 0.2%, about 0.3%, about 0.4%, about 0.5%, about 0.6%, about 0.7%, about 0.5%, about 0.9% or about 1%.

In some embodiments of the invention, the feedstream is at about 4° C. to about 30° C. In other embodiments of the invention, the feedstream is at 4° C. to 30° C. In yet other embodiments, the feedstream is at about 15° C. to about 30° C. In another embodiment, the feedstream is at about 15° C. to about 20° C. In yet further embodiments of the invention, the feedstream is at about 4° C., about 15° C., about 18° C., about 20° C., about 25° C. or about 30° C.

In some embodiments of the invention, the feedstream is at a pH of about 5.5 to about a pH of about 8.0. In other embodiments of the invention, the feedstream is at a pH of 5.5 to a pH of 8.0. In yet further embodiments of the invention, the feedstream is at a pH of about 5.5, about 6.0, about 6.5, about 7.0, about 7.5 or about 8.0.

In some embodiments, the feedstream is incubated with the detergent for less than about 1 minute to about 180 minutes. In other embodiments, the feedstream is incubated with the detergent for about 6 minutes to about 180 minutes. In other embodiments, the feedstream is incubated with the detergent for about 10 minutes to about 180 minutes. In yet other embodiments, the feedstream is incubated with the detergent for about 120 minutes to about 180 minutes. In yet other embodiments, the feedstream is incubated with the detergent for about 180 minutes. In yet other embodiments, the feedstream is incubated with the detergent for about 120 minutes. In yet other embodiments, the feedstream is incubated with the detergent for about 60 minutes. In yet other embodiments, the feedstream is incubated with the detergent for about 1, about 5, about 6, about 10, about 15, about 30, about 45, about 60, about 75, about 90, about 105, about 120, about 135, about 150, about 165, or about 180 minutes.

In embodiments of the invention, the detergent in the feedstream has a viral LRF of greater than about 1. In some embodiments of the invention, the detergent in the feedstream has a viral LRF of greater than about 4. In some embodiments of the invention, the detergent in the feedstream has a viral LRF of greater than about 5. In some embodiments of the invention, the detergent in the feedstream has a viral LRF of greater than about 6. In some embodiments of the invention, the detergent in the feedstream has a viral LRF of greater than about 7. In some embodiments of the invention, the detergent in the feedstream achieves complete viral inactivation. In yet other embodiments of the invention, the detergent in the feedstream has a viral LRF of greater than about 1, about 2, about 3, about 4, about 5, about 6 or about 7.

In some embodiments of the invention, the virus is an enveloped virus. In some embodiments, the virus is a retrovirus, a herpesvirus, a flavivirus, a poxvirus, a hepadnavirus, a hepatitis virus, an orthomyxovirus, a paramyxovirus, a rhabdovirus, or a togavirus.

In some embodiments, the invention provides methods of inactivating virus in a feedstream, the method comprising the step of incubating the feedstream and an environmentally compatible detergent for about 15 minutes to about 180 minutes, at a temperature of about 4° C. to about 30° C. and at a pH of about 5.5 to about 8.0, wherein the detergent is added to the feedstream at a final concentration of about 0.1% w/w to about 1.0% w/w, and wherein the detergent in the feedstream has a viral LRF of greater than about 2.

In some embodiments, the invention provides methods of inactivating virus in a feedstream, the method comprising the step of incubating the feedstream and an environmentally compatible detergent for about 6 minutes to about 180 minutes, at a temperature of about 4° C. to about 30° C. and at a pH of about 5.5 to about 8.0, wherein the detergent is added to the feedstream at a final concentration of about 0.3% w/w to about 1.0% w/w, and wherein the detergent in the feedstream has a viral LRF of greater than about 4.

In some embodiments, the invention provides methods of inactivating virus in a feedstream, the method comprising the step of incubating the feedstream and an environmentally compatible detergent for about 180 minutes, at a temperature of about 4° C. to about 30° C. and at a pH of about 5.5 to about 8.0, wherein the detergent is added to the feedstream at a final concentration of about 0.1% w/w to about 1.0% w/w, and wherein the detergent in the feedstream has a viral LRF of greater than about 5.

In some embodiments, the invention provides methods of inactivating virus in a feedstream, the method comprising the step of incubating the feedstream and an environmentally compatible detergent for about 60 minutes to about 180 minutes, at a temperature of about 4° C. to about 30° C. and at a pH of about 5.5 to about 8.0, wherein the detergent is added to the feedstream at a final concentration of about 0.3% w/w, and wherein the detergent in the feedstream has a viral LRF of greater than about 5.

In some embodiments, the invention provides methods of inactivating virus in a feedstream, the method comprising the step of incubating the feedstream and an environmentally compatible detergent for about 120 minutes to about 180 minutes, at a temperature of about 4° C. to about 30° C. and at a pH of about 5.5 to about 8.0, wherein the detergent is added to the feedstream at a final concentration of about 0.3% w/w, and wherein the detergent in the feedstream has a viral LRF of greater than about 6.

In some embodiments, the invention provides methods of inactivating virus in a feedstream, wherein the feedstream comprises a harvested cell culture fluid, a capture pool or a recovered product pool. In some embodiments, the invention provides methods of inactivating virus in a feedstream, wherein the feedstream comprises a harvested cell culture fluid, a capture pool or a recovered product pool and wherein the capture pool or recovered product pool is an affinity chromatography pool. In yet further embodiments, the capture pool or recovered product pool is a Protein A pool, a Protein G pool or a Protein L pool. In other embodiments, the capture pool or recovered product pool is a mixed mode chromatography pool.

In some embodiments, the invention provides methods wherein the feedstream incubated with the detergent is not subjected to a filtration step. In some embodiments, the invention provides methods wherein the feedstream incubated with the detergent, is subjected to at least one filtration step. In other embodiments, the invention provides methods wherein the feedstream incubated with the detergent is subjected to at least one, or at least two or at least three filtration steps. In some embodiments, the invention provides methods wherein the feedstream incubated with the detergent is subjected to a first filtration step with a 0.22 um filter, followed by a second and a third filtration step with a 0.45 μm PVDF filter. In a specific embodiment, the invention provides methods wherein the filter membrane in the first and/or second and/or the third filtration step comprises, but is not limited to, polyvinylidene difluoride (PVDF), cellulose acetate, cellulose nitrate, polytetrafluoroethylene (PTFE, Teflon), polyvinyl chloride, polyethersulfone, glass fiber filter, or other filter materials suitable for use in a cGMP manufacturing environment. In a preferred embodiment, the invention provides methods wherein the filter membrane in both the second and the third filtration step comprises PVDF. In a specific embodiment, the invention provides methods wherein the filter membrane in both the second and the third filtration step is a membrane with a pore size of about 0.45 μm. In a preferred embodiment, the filter membrane in both the second and the third filtration step comprises a PVDF membrane with a pore size of 0.45 μm. In a specific embodiment, the invention provides methods wherein the filter membrane in the first filtration step is a membrane with a pore size of about 0.22 μm.

In some embodiments, the invention provides methods wherein the feedstream incubated with the detergent is subjected to affinity chromatography. In some embodiments, the affinity chromatography is a Protein A affinity column.

In some embodiments, the invention provides methods wherein the feedstream containing the detergent is subjected to a chromatography column. In some embodiments, the chromatography column is one or more of an affinity column, an ion exchange column, a hydrophobic interaction column, a hydroxyapatite column, or a mixed mode column. In some embodiments, the affinity chromatography column is a Protein A column, a Protein G column or a protein L column. In other embodiments, the ion exchange chromatography column is an anion exchange column or a cation exchange column. In some embodiments, the invention provides methods wherein the detergent is sufficiently removed from the final product. In some embodiments, the invention provides methods wherein the detergent is completely removed from the final product.

In some embodiments, the invention provides methods of inactivating virus in a feedstream, the method comprising the step of adding a detergent to said feedstream and incubating the detergent and feedstream, wherein the therapeutic or diagnostic protein is an antibody, an Fc fusion protein, an immunoadhesin, an enzyme, a growth factor, a receptor, a hormone, a regulatory factor, a cytokine, an antigen, or a binding agent. In further embodiments, the antibody is a monoclonal antibody, a chimeric antibody, a humanized antibody, a human antibody, a bispecific antibody, or an antibody fragment. In some embodiments, the therapeutic or diagnostic protein is produced in mammalian cells. In some embodiments, the mammalian cell is a Chinese Hamster Ovary (CHO) cells, or baby hamster kidney (BHK) cells, murine hybridoma cells, or murine myeloma cells. In some embodiments, the therapeutic or diagnostic protein is produced in bacterial cells. In other embodiments, the therapeutic or diagnostic protein is produced in yeast cells.

In some embodiments of the invention, the feedstream comprising the detergent used in the methods of the present invention is of low turbidity. In some embodiments, addition of the detergent to the feedstream does not significantly increase or change the turbidity of the feedstream. In a further embodiment, the detergent used in the methods of the invention does not affect the product quality of the therapeutic or diagnostic protein.

In some embodiments, the detergent used in the methods of the invention comprises a preservative, and/or a stabilizing agent. In some embodiments, the stabilizing agent is monopropylene glycol. In some embodiments the stabilizing agent in the detergent comprises about 1% to about 5%. In a further embodiment, the stabilizing agent does not affect the viral inactivation properties of the detergent used in the methods of the invention and does not affect the product quality of a therapeutic or diagnostic protein. In a further embodiment, the monopropylene glycol does not affect the viral inactivation properties of the detergent used in the methods of the invention and does not affect the product quality of the therapeutic or diagnostic protein.

In embodiments of the invention, the detergent used in the methods of the invention is environmentally compatible. In embodiments of the invention, the detergent used in the methods of the invention does not comprise or form 4-(1,1,3,3-tetramethylbutyl) phenol (aka 4-tert-octylphenol). In other embodiments, the detergent used in the methods of the invention does not comprise or form peroxide. In other embodiments of the invention, the detergent used in the methods of the invention is biodegradable.

In one aspect of the invention, the invention provides a method of inactivating a virus in a feedstream comprising the steps of:

adding to the feedstream an undecyl alkyl glycoside at a concentration of about 0.1% w/w to about 0.3% w/w;

incubating the undecyl alkyl glycoside in the feedstream for about 180 minutes, wherein the viral log reduction factor (LRF) of the undecyl alkyl glycoside in the feedstream is greater than about 5;

filtering said feedstream containing the undecyl alkyl glycoside; and subjecting said filtered feedstream to a Protein A affinity chromatography column;

wherein at said step of adding and incubating, the feedstream is at a temperature of about 4° C. to about 30° C. and at a pH of about 5.5 to about 8.0.

In one aspect of the invention, the invention provides a method of inactivating a virus in a feedstream comprising the steps of:

adding to the feedstream SIMULSOL™ SL 11W at a concentration of about 0.1% w/w to about 0.3% w/w;

incubating the SIMULSOL™ SL 11W in the feedstream for about 180 minutes, wherein the viral log reduction factor (LRF) of SIMULSOL™ SL 11W in the feedstream is greater than about 5;

filtering said feedstream containing the SIMULSOL™ SL 11W; and subjecting said filtered feedstream to a Protein A affinity chromatography column;

wherein, at said step of adding and incubating, the feedstream is at a temperature of about 4° C. to about 30° C. and at a pH of about 5.5 to about 8.0. In yet a further aspect of the invention, the filtration step in the methods of the invention comprises a first filtration step with a 0.22 μm filter. In yet another aspect, the filtration step in the methods of the invention comprises a second filtration step with a 0.45 PVDF filter. In yet another aspect, the filtration step in the methods of the invention comprises a third filtration step with a 0.45 μm PVDF filter. In a further embodiment, the filter membrane in the first and/or second and/or the third filtration step in the methods of the invention comprises, but is not limited to, polyvinylidene difluoride (PVDF), cellulose acetate, cellulose nitrate, polytetrafluoroethylene (PTFE, Teflon), polyvinyl chloride, polyethersulfone, glass fiber filter, or other filter materials suitable for use in a cGMP manufacturing environment. In a preferred embodiment, the filter membrane comprises PVDF. In a specific embodiment, the filter membrane in both the second and the third filtration step is a membrane with a pore size of about 0.45 μm. In a preferred embodiment, the filter membrane is a PVDF membrane with a pore size of 0.45 μm.

As used herein, an “environmentally compatible” detergent or compound or substance or agent, as referred to herein, causes minimal and/or acceptable levels of harmful effects on the environment. For example, the Organization for Economic Co-operation and Development (“OECD”) provides guidelines for testing chemical safety. Embodiments of environmentally compatible agents according to the present disclosure may also be biodegradable. Methods to determine whether an agent such as a detergent is environmentally compatible for example, under the conditions for protein manufacturing are known in the art. Environmental compatibility may also be assessed by ecological risk assessment, which is the process for evaluating how likely it is that the environment may be impacted as a result of exposure to one or more environmental stressors such as chemicals, land change, disease, invasive species and climate change. These guidelines can be found, for example, on the United States Environmental Protection Agency website. Additionally, guidelines on registration, evaluation, authorization and restriction of chemicals in the EU can be found on the European Commission REACH website.

A “predicted environmental concentration” or “PEC” is the predicted concentration of a substance in waste material discharged into the receiving water body in environment. For example, a predicted environmental concentration of a detergent used for viral inactivation in the preparation of a therapeutic or diagnostic protein is the concentration of detergent in the waste stream that is discharged into the environment.

The term “detergent” as used herein, refers to an agent that may comprise salts of long-chain aliphatic bases or acids, or hydrophilic moieties such as sugars, and that possess both hydrophilic and hydrophobic properties. As used herein, detergents can have the ability to disrupt viral envelopes and inactivate viruses. The mechanism of virus inactivation by detergents is attributed to interactions between the detergent and the lipid components of the virus outer membrane, which result in the disruption of the membrane and ultimately loss of virulence. In some examples, a detergent may be a composition comprising a “surfactant” or “surface acting agent” and one or more other agents such as chelating agents, preservatives or stabilizing agents. Detergents or surfactants may be classified based upon charge. Surfactants can be non-ionic, cationic or anionic. As used herein, a surfactant has the ability to disrupt viral envelopes and inactivate viruses. Embodiments of detergents, according to the present disclosure, comprise an alkyl glycoside, wherein the alkyl glycoside can be an alkyl glucoside.

“Alkyl glycoside” according to the present disclosure, refers to any sugar joined by a linkage to any hydrophobic alkyl. “Alkyl glucoside” as used herein, is comprised of an alkyl group linked to a sugar, wherein the sugar is a glucose. An alkyl glycoside may be, for example, an undecyl alkyl glycoside. An undecyl alkyl glycoside may comprise an undecyl chain with O-glycosidic linkage to mono-D-glucopyranose (for example, n-undecyl-β-D-glucopyranose), or D-glucopyranose oligo- or polysaccharides, or mixtures thereof. The chemical synthesis of alkyl glycosides such as those used in the methods of the present invention may result in a heterogeneous mixture of compounds, rather than a completely homogeneous preparation. As such, unless otherwise noted, references used herein to a particular form of alkyl glycoside, means that at least the majority component of any heterogeneous mixture is that form of alkyl glycoside, such as an undecyl alkyl glycoside. Examples of, such alkyl glycosides, which comprise undecyl alkyl glycosides include, but are not limited, to SIMULSOL™ SL 11W and SIMULSOL™ SL 82.

A “product feedstream” or “feedstream” is the material or solution provided for a process purification method which contains a therapeutic or diagnostic protein of interest and which may also contain various impurities. Non-limiting examples may include, for example, harvested cell culture fluid (HCCF), harvested cell culture material, clarified cell culture fluid, clarified cell culture material, the capture pool, the recovered pool, and/or the collected pool containing the therapeutic or diagnostic protein of interest after one or more centrifugation steps, and/or filtration steps, the capture pool, the recovered protein pool and/or the collected pool containing the therapeutic or diagnostic protein of interest after one or more purification steps.

The term “impurities” refers to materials that are different from the desired protein product. The impurity includes, without limitation: host cell materials, such as CHOP; leached Protein A; nucleic acid; a variant, size variant, fragment, aggregate or derivative of the desired protein; another protein; endotoxin; viral contaminant; cell culture media component, etc.

The terms “protein” and “polypeptide” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, proteins containing one or more analogs of an amino acid (including, for example, unnatural amino acids, etc.), as well as other modifications known in the art. Examples of proteins include, but are not limited to, antibodies, peptides, enzymes, receptors, hormones, regulatory factors, antigens, binding agents, cytokines, Fc fusion proteins, immunoadhesin molecules, etc.

The term “antibody” or “antibodies” is used in the broadest sense and specifically covers, for example, monoclonal antibodies (including agonist, antagonist, and neutralizing antibodies), chimeric antibodies, humanized antibodies, human antibodies, antibody compositions with polyepitopic specificity, polyclonal antibodies, single chain antibodies, multi specific antibodies (e.g., bispecific antibodies), immunoadhesins, and fragments of antibodies as long as they exhibit the desired biological or immunological activity. The term “immunoglobulin” (Ig) is used interchangeably with antibody herein.

The term “ultrafiltration” or “filtration” is a form of membrane filtration in which hydrostatic pressure forces a liquid against a semipermeable membrane. Suspended solids and solutes of high molecular weight are retained, while water and low molecular weight solutes pass through the membrane. In some examples, ultrafiltration membranes have pore sizes in the range of 1 μm to 100 μm. The terms “ultrafiltration membrane” “ultrafiltration filter” “filtration membrane” and “filtration filter” may be used interchangeably. Examples of filtration membranes include but are not limited to polyvinylidene difluoride (PVDF) membrane, cellulose acetate, cellulose nitrate, polytetrafluoroethylene (PTFE, Teflon), polyvinyl chloride, polyethersulfone, glass fiber, or other filter materials suitable for use in a cGMP manufacturing environment.

As used herein, numeric ranges are inclusive of the numbers defining the range.

The term “enveloped virus”, or “lipid-coat containing virus” refers to any virus comprising a membrane or envelope including lipid, such as, e.g., an envelope virus. Enveloped viruses have their capsid enclosed by a lipoprotein membrane, or envelope. This envelope is derived from the host cell as the virus “buds” from its surface and consists mostly of lipids not encoded by the viral genome. Even though it carries molecular determinants for attachment and entry into target cells, and is essential for the infectivity of enveloped viruses, it is not subject to drug resistance or antigenic shift. Enveloped viruses range in size from about 45-55 nm to about 120-200 nm. Non-limiting examples of lipid-coat containing viruses which can infect mammalian cells include DNA viruses like a herpesviridae virus, a poxviridae virus, or a hepadnaviridae virus; RNA viruses like a flaviviridae virus, a togaviridae virus, a coronaviridae virus, a deltavirus virus, an orthomyxoviridae virus, a paramyxoviridae virus, a rhabdoviridae virus, a bunyaviridae virus, or a filoviridae virus; and reverse transcribing viruses like a retroviridae virus or a hepadnaviridae virus. In a variation, the invention provides methods for inactivating a subviral agent in a product feedstream comprising subjecting the feedstream to an environmentally compatible detergent. In some aspects, the subviral agent is a viroid or a satellite. In another variation, the invention provides methods for inactivating a virus-like agent in a product feedstream comprising subjecting the feedstream to an environmentally compatible detergent.

Methods to measure viral activity or viral infectivity are known in the art. Examples include, but are not limited to, TCID₅₀ assays (i.e., determination of the median tissue culture infective dose that will produce pathological change in 50% of cell cultures inoculated) and plaque assays. Other known methods may include, for example, transformation assay, which can be used to determine the titers of the biological activity of non-plaque forming viruses that have the ability to cause cellular growth transformation; or the fluorescent-focus assay which relies on the use of antibody staining methods to detect virus antigens within infected cells in the monolayer, or the endpoint dilution assay which can be used to determine the titers of many viruses, including viruses which do not infect monolayer cells (as an alternative to plaque assays); or viral enzyme assays in which virally-encoded enzymes such as reverse transcriptases or viral proteases are measured. Furthermore, detection of a specific virus can be accomplished by polymerase chain reaction (PCR) using primers and probes designed to detect a specific virus.

Inactivation of virus in a product feedstream can be measured using methods known in the art. In some embodiments of the invention, viral inactivation is expressed as log reduction factor (“LRF”) or log reduction value (“LRV”). LRF is calculated as:

$$R = {log}_{10} \frac{{V}_{i} {T}_{i}}{{V}_{o} {T}_{o}}$$

Where, R=the log reduction factor (LRF), Vᵢ=the input volume in mL, Tᵢ=the input virus titer, Vₒ=the output volume and Tₒ=the output virus titer.

### EXAMPLES

### Example 1. Viral Inactivation with Non-Ionic Glycoside Detergents

### Detergents and Reagents

Stock solution for the exemplified detergents listed in Table 1 (Seppic Inc. Fairfield, N.J.), and 1,2-propanediol (Sigma-Aldrich, St. Louis, Mo.) are prepared in water on a weight/volume (w/w) percentage.

TABLE 1
Detergents Types

| | | Physical |
|---|---|---|
| Detergent Name | Alkyl glycoside content | Properties |
| SIMULSOL ™ | D-Glucopyranose, oligomeric, undecyl glycoside (40-60%), | Clear |
| SL 11W | propane-1,2-diol (1-5%) | Liquid |
| SIMULSOL ™ | 2 ethylhexyl mono-D-glycopyranoside, 2 ethylhexyl di-D- | White Soft |
| AS 48 | glycopyranoside (40-60%) | Solid |
| SIMULSOL ™ | D-Glucopyranose, oligomeric, butyl glycoside (40-60%) | Clear |
| SL 4 | | Liquid |
| SIMULSOL ™ | D-Glucopyranose, oligomeric, C10-16 (even numbered)-alkyl | White Soft |
| SL 10 | glycosides (40-60%) | Solid |
| SIMULSOL ™ | D-Glucopyranose, oligomeric, C10-16 (even numbered)-alkyl | White Soft |
| SL 26 C | glycosides (40-60%) | Solid |
| SIMULSOL ™ | D-Glucopyranose, oligomeric, undecyl glycoside (40-60%), | Clear |
| SL 82 | D-Glucopyranose, oligomeric, C10-16 (even numbered)-alkyl | Liquid |
| | glycosides (10-20%), propane-1,2-diol (1-5%) | |
| SIMULSOL ™ | D-Glucopyranose, oligomers, decyl octyl glycosides (40- | Clear |
| SL 8 | 60%) | Liquid |
| SIMULSOL ™ | D-Glucopyranose, oligomers, decyl octyl glycosides (20- | Clear |
| SL 826 | 40%), D-glucopyranose, oligomeric, C10-16 (even | Liquid |
| | numbered)-alkyl glycosides (20-40%), (2- | |
| | methoxymethylethoxy)propanol (0.1-1%) | |

### Viruses and Indicator Cells

Xenotropic Murine Leukemiavirus (XMuLV), Porcine parvovirus (PPV) NADL-2 strain and pseudorabies virus (PRV) for use in these studies may be purchased from ATCC, Manassas, Va. Table 2 shows the properties of these viruses.

TABLE 2
Virus characteristics

| | | | | Physio- | |
|---|---|---|---|---|---|
| | | | Size | chemical | Envelope |
| Virus | Genome | Genus | (nm) | Resistance | (Yes or No) |
| XMuLV | DNA | Retroviridae | 80-100 | Low | Yes |
| PRV | RNA | Herpesviradae | 120-140 | Low | Yes |
| PPV | DNA | Parvoviradae | 18-22 | High | No |

PK13 (pig epithelial cells), PG-4 (feline brain cells) and Vero (African Green Monkey cells) for use in these studies may be purchased from ATCC, Manassas, Va. PK-13 cells are cultured in Dulbecco's Modified Eagle's Medium (DMEM, Gibco, Grand Island, N.Y.). PG-4 cells are cultured in McCoy's 5a Media (ATCC, Manassas, Va.) and Vero cells are cultured in Earl's modified Eagle's Medium (EMEM, Gibco, Grand Island, N.Y.). All cells are supplemented with 10% FBS (HyClone, Logan, Utah).

Cell based TCID₅₀ Virus Titration Assay

A cell-based tissue culture infectious dose 50 (TCID₅₀) assay is used for viral titration for XMuLV with PG-4 indicator cells, PPV with PK-13 indicator cells and PRV with Vero indicator cells. 96-well tissue culture plates are seeded with 2.5×10⁴ cells/mL indicator cells at 200 μL/well and incubated at 37° C. overnight or for about 16-24 hours. The plates are then inoculated with the virus in a series of 10-fold dilutions at 50 μL/well at a minimum of n=8 per dilution. The inoculated plates are incubated for 7-9 days (XMuLV and PRV) and 10-13 days (PPV) at 37° C. The plates are then examined and scored as positive or negative well-by-well under a microscope for cytopathic effects (CPE), and viral titers are calculated by standard methods.

### Viral Cytotoxicity and Interference in Clarified Bioreactor Harvest Material

The TCID₅₀ assay exemplified above is used to determine the cytotoxic effects of viruses in clarified bioreactor harvest material of monoclonal antibody (mAb), bispecific antibody and Fc-fusion proteins. The bioreactor harvest materials are clarified by centrifugation followed by polishing filtration. Impurities such as HCPs, lipids and host cell DNA are also automatically concentrated as part of the centrifugation process. The clarified bioreactor harvest material for the different proteins containing from 0.05% w/w to 1% w/w detergents are diluted in inoculation media at 10, 50 and 100-fold. Each dilution is added at n=8 to 96 well indicator cell seeded plates and incubated as per the TCID₅₀ assay. At the end of the incubation period, each well is observed under a microscope for CPE and the appropriate dilution needed for each detergent is determined. The results demonstrated that a 50-fold dilution factor was sufficient to overcome cytotoxicity by the detergent.

The clarified bioreactor harvest materials for the various protein types are evaluated for any intrinsic effects of the cell culture harvest material on virus replication. Each virus is diluted 10-fold in the clarified bioreactor harvest material and inoculated onto indicator cell seeded plates and the experiment is conducted as per the TCID₅₀ assay. A positive control (virus spiked into tissue culture material) is included. The viral titers are compared to the positive control virus. Titers that are within ±0.5 log₁₀ of the positive control titers are considered to have no viral interference occurring. All viral test titers fell within ±0.5 log₁₀ of the positive control titers and thus no viral interference was observed for any of the bioreactor harvest materials, regardless of protein type.

### Viral Inactivation General Procedure

Virus inactivation of different enveloped viruses by each detergent is evaluated at a range of different concentrations, temperatures, and pH, in clarified bioreactor harvest materials containing mAb, bispecific and Fc-fusion proteins. XMuLV, PPV or PRV viruses are spiked into the harvest material at no more than 5% w/w, and detergent stock solution is added to the spiked material to achieve a specified final detergent concentration. The samples are then incubated at specific temperature and pH ranges. The viral inactivation is initiated as soon as the detergent is added to the virus containing material. The kinetics of virus inactivation are monitored by removing aliquots at various time points from the harvest samples and viral titer are assessed by the TCID₅₀ assay. Matrix controls are included in the studies to demonstrate that virus inactivation is a result of detergent treatment and not the matrix itself.

### XMuLV Inactivation at 30° C. by Non-Ionic Detergents

Viral inactivation of XMuLV by the detergents listed in Table 1 is tested at the optimal conditions determined for XMuLV inactivation. As per the Viral Inactivation Procedure exemplified above, each detergent is added at 0.6% w/w to the XMuLV spiked clarified mAb bioreactor harvest material at 30° C. Viral inactivation is monitored at various timepoints with a maximum time of inactivation of 180 minutes post virus inoculation.

The results compiled in Table 3, show that at the optimal conditions for XMuLV inactivation, the XMuLV is rapidly and completely inactivated for all the detergents tested at 180 minutes except for SIMULSOL™ SL 4 and SIMULSOL™ AS 48. Since there was no virus inactivation observed with SIMULSOL™ SL 4, the concentration of SIMULSOL™ SL 4 is increased to 30 mM (1%) and XMuLV inactivation is monitored at 30° C. for 180 minutes. However, no XMuLV inactivation was observed at the increased concentration of SIMULSOL™ SL 4 (data not shown). Surprisingly, these results suggest that not all glycosides are capable of robust viral inactivation, or they require extremely high concentrations to achieve robust inactivation as was observed for SIMULSOL™ AS 48. However, SIMULSOL™ SL 11W showed complete viral inactivation of greater than an LRF of 4, within 0 to 5 minutes of adding it to the clarified mAb bioreactor harvest material. Furthermore, even though the other glycosides effectively inactivated XMuLV, other factors limit applicability of some of these detergents in a large-scale manufacturing process. Such detergent characteristics included solubility, effect on turbidity in the harvested material, activity at different temperature and pH ranges, unknown or limited data on environmental toxicity and ease of handling.

TABLE 3
XMuLV Inactivation at 30° C. by Non-Ionic Detergents
in clarified mAb bioreactor harvest material

| Detergent | LRF Achieved at 30° C. | | |
|---|---|---|---|
| (0.6% w/w) | 0-5 min | 90 min | 180 min |
| SIMULSOL ™ SL 11W | ≥4.55 | ≥4.55 | ≥4.55 |
| SIMULSOL ™ AS 48 | 0.50 ± 0.56 | 2.13 ± 0.56 | 3.20 ± 0.44 |
| SIMULSOL ™ SL 4 | 0.88 ± 0.40 | 0.38 ± 0.50 | 0.88 ± 0.40 |
| SIMULSOL ™ SL 10 | ≥3.10 | ≥4.62 | ≥4.62 |
| SIMULSOL ™ SL 26 C | ≥3.60 | ≥5.12 | ≥5.12 |
| SIMULSOL ™ SL 82 | ≥3.60 | ≥5.12 | ≥5.12 |
| SIMULSOL ™ SL 8 | ≥3.10 | ≥4.62 | ≥4.62 |
| SIMULSOL ™ SL 826 | ≥3.10 | ≥4.62 | ≥4.62 |

### Turbidity of SIMULSOL™ SL 11W in Clarified mAb Bioreactor Harvest Material.

Turbidity is evaluated at SIMULSOL™ SL 11W concentrations of 0%, 0.05%, 0.1%, 0.3%, 0.5% and 1% w/w in XMuLV spiked into clarified mAb bioreactor harvest material at 18° C. Viral inactivation is monitored for 180 mins and the turbidity is measured in nephelometric turbidity units (NTU) using a portable turbidimeter 2100P (Hach®).

The results as demonstrated in Table 4, show that SIMULSOL™ SL 11W at concentrations ranging from 0% to 1% w/w, was stable over time and no gross precipitation was observed in the harvest material up to 180 minutes.

TABLE 4
Turbidity of SIMULSOL ™ SL 11W in clarified mAb bioreactor
harvest material incubated at 18° C.

| % w/w | Turbidity (NTU) | |
|---|---|---|
| SIMULSOL ™ | Time 0 (2 | Time 180 (2 |
| SL 11W | measurements) | measurements) |

| | | | | |
|---|---|---|---|---|
| 0 | 7.92 | 7.92 | 7.6 | 7.59 |
| 0.05 | 11.61 | 11.6 | 15.7 | 16.2 |
| 0.1 | 51.5 | 52.1 | 49.4 | 49.1 |
| 0.3 | 75.8 | 75 | 91.6 | 91.5 |
| 0.5 | 355 | 357 | 377 | 375 |
| 1 | 834 | 836 | 754 | 760 |

### Characterization and Optimization of Viral Inactivation by SIMULSOL™ SL 11W

The use of SIMULSOL™ SL 11W as a viable environmentally compatible detergent for use in the manufacturing processes of proteins is further evaluated in clarified mAb bioreactor harvest material as per the Viral Inactivation Procedure exemplified above. Briefly, SIMULSOL™ SL 11W activity is evaluated at concentrations of 0.05%, 0.1%, 0.3%, 0.5% and 1% w/w in XMuLV spiked clarified mAb bioreactor harvest material at 4 different temperatures of 4° C., 18° C., 25° C., and 30° C. Viral inactivation is monitored at 0, 10, 30, 60, 120- and 180-min post inoculation with XMuLV to the SIMULSOL™ SL 11W containing clarified mAb bioreactor harvest material. The results as demonstrated in Tables 5-9, show that SIMULSOL™ SL 11W concentration, incubation time and temperature are all factors that influence the inactivation of XMuLV. SIMULSOL™ SL 11W achieved complete XMuLV viral inactivation at concentrations ≥0.1% w/w (≥3 mM) by 120 min and at concentrations ≥0.3% w/w by 10 minutes at all 4 temperatures evaluated.

TABLE 5
XMuLV Inactivation by 1.5 mM (0.05%) SIMULSOL ™
SL 11W in clarified mAb bioreactor harvest material

| | |
|---|---|
| Time Point | LRF Achieved |

| (min) | 4° C. | 18° C. | 25° C. | 30° C. |
|---|---|---|---|---|
| 0 | −0.38 | 0.13 | 0.13 | 0.25 |
| 10 | −0.13 | 0.00 | 0.13 | 0.75 |
| 30 | 0.50 | 1.38 | 1.88 | 1.37 |
| 60 | 0.62 | 2.25 | 2.00 | 1.37 |
| 120 | 1.25 | 2.25 | 2.59 | 1.75 |
| 180 | 2.12 | 3.94 | 3.33 | 2.12 |

TABLE 6
XMuLV Inactivation by 3.0 mM (0.1%) SIMULSOL ™
SL 11W in clarified mAb bioreactor harvest material

| | |
|---|---|
| Time Point | LRF Achieved |

| (min) | 4° C. | 18° C. | 25° C. | 30° C. |
|---|---|---|---|---|
| 0 | 0.62 | 3.25 | 2.12 | 3.25 |
| 10 | 2.50 | 3.41 | 2.55 | 3.50 |
| 30 | 4.58 | 3.42 | 2.71 | 4.08 |
| 60 | 5.43 | 4.99 | 3.64 | 5.00 |
| 120 | ≥5.42 | ≥5.92 | ≥4.92 | ≥6.05 |
| 180 | ≥5.88 | ≥6.38 | ≥5.38 | ≥6.51 |

TABLE 7
XMuLV Inactivation by 9.0 mM (0.3%) SIMULSOL ™
SL 11W in clarified mAb bioreactor harvest material

| | |
|---|---|
| Time Point | LRF Achieved |

| (min) | 4° C. | 18° C. | 25° C. | 30° C. |
|---|---|---|---|---|
| 0 | 1.50 | 3.21 | 2.25 | ≥5.17 |
| 10 | ≥5.13 | ≥5.05 | ≥5.05 | ≥5.17 |
| 30 | ≥5.30 | ≥5.05 | ≥5.05 | ≥5.17 |
| 60 | ≥5.30 | ≥5.05 | ≥5.05 | ≥5.17 |
| 120 | ≥5.30 | ≥5.05 | ≥5.05 | ≥5.17 |
| 180 | ≥5.76 | ≥5.51 | ≥5.51 | ≥5.63 |

TABLE 8
XMuLV Inactivation by 15 mM (0.5%) SIMULSOL ™ SL
11W in clarified mAb bioreactor harvest material

| | |
|---|---|
| Time Point | LRF Achieved |

| (min) | 4° C. | 18° C. | 25° C. | 30° C. |
|---|---|---|---|---|
| 0 | 2.61 | 5.36 | ≥5.05 | ≥4.55 |
| 10 | ≥4.67 | ≥5.05 | ≥5.05 | ≥4.55 |
| 30 | ≥4.67 | ≥5.05 | ≥5.05 | ≥4.55 |
| 60 | ≥4.67 | ≥5.04 | ≥5.05 | ≥4.55 |
| 120 | ≥4.67 | ≥5.05 | ≥5.05 | ≥4.55 |
| 180 | ≥5.13 | ≥5.51 | ≥5.51 | ≥5.01 |

TABLE 9
XMuLV Inactivation by 30 mM (1.0%) SIMULSOL ™ SL
11W in clarified mAb bioreactor harvest material

| | |
|---|---|
| Time Point | LRF Achieved |

| (min) | 4° C. | 18° C. | 25° C. | 30° C. |
|---|---|---|---|---|
| 0 | 2.91 | 5.61 | ≥4.67 | ≥5.42 |
| 10 | ≥5.42 | ≥5.30 | ≥4.67 | ≥5.42 |
| 30 | ≥5.42 | ≥5.30 | ≥4.67 | ≥5.42 |
| 60 | ≥5.42 | ≥5.30 | ≥4.67 | ≥5.42 |
| 120 | ≥5.42 | ≥5.30 | ≥4.67 | ≥5.42 |
| 180 | ≥5.88 | ≥5.76 | ≥5.13 | ≥5.88 |

### XMuLV Inactivation by SIMULSOL™ SL 11W in Different Starting Matrices

The ability of SIMULSOL™ SL 11W to inactivate virus at 9 mM (0.3% w/w) in different starting matrices such as water, cell culture media and Dulbecco's Phosphate Buffered Saline (DPBS) at 4° C. and 30° C. is evaluated. Viral inactivation is monitored at 0, 60-, 120- and 180-minutes post inoculation with XMuLV.

The results as demonstrated in Table 10, show robust XMuLV inactivation at both 4° C. and 30° C. after 120 minutes in all three starting matrices. The DPBS at 4° C. after 180 minutes detected a few live viral particles in the TCID₅₀ assay but an LRF of 5.63 was still achieved. Overall, these results suggest that SIMULSOL™ SL 11W is capable of effectively inactivating virus independent of starting matrix. This suggests the potential use of SIMULSOL™ SL 11W across biopharmaceutical manufacturing platforms and the flexibility of the use of SIMULSOL™ SL 11W at different stages of the manufacturing process of a protein.

TABLE 10
XMuLV inactivation by SIMULSOL ™
SL 11W in different starting matrices

| | LRF (0.3% w/w SIMULSOL ™ SL 11W) | | | | | |
|---|---|---|---|---|---|---|
| | 4° C. | | | 30° C. | | |
| Time | | Cell culture | | | Cell culture | |
| (min) | Water | media | DPBS | Water | media | DPBS |
| 0 | 4.12 | 2 | 1.5 | ≥4.40 | ≥4.40 | 4.41 |
| 60 | ≥4.52 | ≥4.52 | 4.53 | ≥4.40 | ≥4.40 | ≥4.40 |
| 120 | ≥4.52 | ≥4.52 | ≥4.52 | ≥4.40 | ≥4.40 | ≥4.40 |
| 180 | ≥5.31 | ≥5.31 | 5.63 | ≥5.05 | ≥5.05 | ≥5.05 |

### XMuLV Inactivation Kinetics at 0.3% SIMULSOL™ SL 11W at 4° C. and 30° C.

The ability of SIMULSOL™ SL 11W to inactivate virus at lower timepoints is evaluated. SIMULSOL™ SL 11W is added at 9 mM (0.3% w/w) to clarified mAb bioreactor harvest material at 4° C. and 30° C. Virus inactivation is monitored at 0, 2, 4, 6, 8, 10 and 20- and 180-minutes post inoculation with XMuLV.

The results as demonstrated in Table 11, show complete XMuLV inactivation occurring at ≥6 mins at 4° C., and at ≥2 mins at 30° C. with 9 mM (0.3% w/w) SIMULSOL™ SL 11W in clarified mAb bioreactor harvest material.

TABLE 11
XMuLV inactivation by 9 mM (0.3%) SIMULSOL ™ SL 11W at 4° C.
and 30° C. in clarified mAb bioreactor harvest material

| | |
|---|---|
| Time Point | LRF Achieved |

| (minutes) | 4° C. | 30° C. |
|---|---|---|
| 0 | 1.75 | 3.62 |
| 2 | 3.32 | ≥5.05 |
| 4 | 4.27 | ≥5.05 |
| 6 | ≥4.92 | ≥5.05 |
| 8 | ≥4.92 | ≥5.05 |
| 10 | ≥4.92 | ≥5.05 |
| 20 | ≥4.92 | ≥5.05 |
| 180 | ≥5.38 | ≥5.51 |

### Effects of Stabilizing Agent Monopropylene Glycol on Virus Inactivation

The effect of monopropylene glycol on viral inactivation at concentration of 0.1% and 0.5% in clarified mAb bioreactor harvest materials at 30° C. is evaluated. Viral inactivation is monitored at 0 and 180 mins post inoculation with XMuLV. SIMULSOL™ SL 11W contains 1% to 5% monopropylene glycol. The maximum concentration of monopropylene glycol found in the inactivation procedures reported herein is 0.05%. The results as demonstrated in Table 12, show that monopropylene glycol at concentrations of 0.1% and 0.5% id not inactivate XMuLV in clarified mAb bioreactor harvest material at 30° C. Furthermore, the concentrations of monopropylene glycol demonstrated in Table 11 are at least 10-fold above the maximum concentration that would be found in clarified mAb bioreactor harvest material treated with SIMULSOL™ SL 11W. This indicates that the SIMULSOL™ SL 11W is responsible for the observed virus inactivation

TABLE 12
XMuLV inactivation by monopropylene glycol in
clarified mAb bioreactor harvest material

LRF at 30° C.

| | 0.1% | 0.5% |
|---|---|---|
| Time Point | monopropylene | monopropylene |
| (minutes) | glycol | glycol |
| 0 | 0.25 | 0.00 |
| 180 | 0.25 | 0.13 |

### Enveloped and Non-Enveloped Viral Inactivation by SIMULSOL™ SL 11W

The ability of SIMULSOL™ SL 11W to inactivate PRV, an enveloped virus and PPV a non-enveloped virus is evaluated. SIMULSOL™ SL 11W is added at 9 mM (0.3% w/w) to clarified mAb bioreactor harvest at 4° C., 18° C. and 30° C. Viral inactivation is monitored at 0, 10, 30, 60, 120- and 180-minutes post inoculation

The results as demonstrated in Table 13, shows robust inactivation of PRV enveloped virus occurring by 10 minutes at all three temperatures, with robust inactivation of PRV enveloped virus occurring at even lower time points within 0 to 5 mins at 30° C. No inactivation of PPV was observed by SIMULSOL™ SL 11W. These results taken together suggest that SIMULSOL™ SL 11W not only robustly inactivates different enveloped viruses in clarified mAb bioreactor harvest materials, but that viral inactivation by SIMULSOL™ SL 11W is specific to enveloped viruses.

TABLE 13
PRV Inactivation by 9 mM (0.3% w/w) SIMULSOL ™
SL 11W in clarified mAb bioreactor harvest material

| Time point | LRF Achieved | | |
|---|---|---|---|
| (min) | 4° C. | 18° C. | 30° C. |
| 0-5 | 2.93 | 1.30 | ≥3.30 |
| 10 | ≥2.92 | ≥2.92 | ≥3.30 |
| 30 | ≥2.92 | ≥3.23 | ≥3.30 |
| 60 | ≥2.92 | ≥2.92 | ≥3.30 |
| 120 | ≥2.92 | ≥2.92 | ≥3.30 |
| 180 | ≥3.38 | ≥3.38 | ≥3.76 |

Effect of pH on XMuLV Inactivation with SIMULSOL™ SL 11W

The ability of SIMULSOL™ SL 11W to inactivate XMuLV at a pH of 5.5 and a pH of 8.0 is evaluated. SIMULSOL™ SL 11W is added at 9 mM (0.3% w/w) to clarified mAb bioreactor harvest material at 18° C. Viral inactivation is monitored at 0, 10, 30, 60, 120- and 180-minutes post inoculation with XMuLV to the SIMULSOL™ SL 11W containing clarified mAb bioreactor harvest material.

The results as demonstrated in Table 14, show rapid and complete inactivation of XMuLV by SIMULSOL™ SL 11W at both pH 5.5 and pH 8.0 by 10 mins, thus indicating potential applicability of SIMULSOL™ SL 11W over a broad pH range in the manufacturing process of proteins.

TABLE 14
XMuLV inactivation by 9 mM (0.3%) SIMULSOL ™
SL 11W at 18° C., pH 5.5 and pH 8.0 in clarified
mAb bioreactor harvest material

| | |
|---|---|
| Time Point | LRF Achieved |

| (min) | pH 5.5 | pH 8.0 |
|---|---|---|
| 0 | 3.48 | 3.47 |
| 10 | ≥5.42 | ≥5.42 |
| 30 | ≥5.42 | ≥5.42 |
| 60 | ≥5.42 | ≥5.42 |
| 120 | ≥5.42 | ≥5.42 |
| 180 | ≥5.88 | ≥5.88 |

Effect of Bioreactor Harvest Material Source on Inactivation of XMuLV with SIMULSOL™ SL 11W

The ability of SIMULSOL™ SL 11W to inactivate XMuLV in clarified bioreactor harvest materials containing different recombinant proteins such as mAb, bispecific antibody, and Fc fusion protein is evaluated. SIMULSOL™ SL 11W is added at 9 mM (0.3% w/w) to the clarified bioreactor harvest materials of the different proteins at 4° C. and 18° C. Viral inactivation is monitored at incubation time points of 0, 10, 30, 60, 120 and 180 minutes. The results as demonstrated in Table 15, show complete XMuLV inactivation by SIMULSOL™ SL 11W by 120 mins at both 4° C. and 30° C. in all three clarified bioreactor harvest materials. Further, complete inactivation was observed in the Fc-fusion and mAb protein containing harvest material by 10 mins at 4° C. The inactivation kinetics for the bispecific antibody was slightly slower at 4° C. than the other two clarified bioreactor harvest materials at 4° C. However, at 18° C., complete inactivation by SIMULSOL™ SL 11W was observed for all 3 proteins within 10 mins.

TABLE 15
Virus inactivation by 9 mM (0.3%) SIMULSOL ™ SL 11W in
clarified recombinant protein bioreactor harvest materials

| Time | 4° C. | | | 18° C. | | |
|---|---|---|---|---|---|---|
| point | Fc Fusion | Bispecific | | Fc Fusion | Bispecific | |
| (min) | protein | antibody | mAb | protein | antibody | mAb |
| 0 | 2.96 | 1.87 | 1.50 | 3.55 | 2.25 | ≥5.17 |
| 10 | ≥5.17 | 2.55 | ≥5.13 | ≥5.17 | ≥4.92 | ≥5.17 |
| 30 | ≥5.17 | 3.27 | ≥5.30 | ≥5.17 | ≥4.92 | ≥5.17 |
| 60 | ≥5.17 | 4.32 | ≥5.30 | ≥5.17 | ≥4.92 | ≥5.17 |
| 120 | ≥5.17 | ≥4.92 | ≥5.30 | ≥5.17 | ≥4.92 | ≥5.17 |
| 180 | ≥5.63 | ≥5.38 | ≥5.76 | ≥5.63 | ≥5.38 | ≥5.63 |

### Effect of Impurity Levels on Viral Inactivation in Clarified Bioreactor Harvest Material of Different Proteins

The effect of impurity levels in the bioreactor harvest materials for three different recombinant proteins: an Fc fusion protein, a bispecific antibody, and a mAb are evaluated.

The harvest materials are clarified by centrifugation followed by polishing filtration. Impurities such as HCPs, lipids and host cell DNA are also automatically concentrated as part of the centrifugation process. A sample of the clarified mAb harvest material is further concentrated about 4-fold through a 5 KD molecular weight cutoff tangential flow filtration (TFF) process. The recombinant protein, DNA and other HCP impurities in the three clarified materials and the TFF concentrated material are measured. The results as demonstrated in Table 16, show the recombinant protein, host cell DNA and HCP content profiles in the three recombinant protein clarified materials and the concentrated clarified mAb material. A greater than 3-fold increase in DNA and HCP impurities was observed in the concentrated clarified mAb harvest material when compared to the unconcentrated clarified mAb harvest material.

TABLE 16
Measure of components in clarified recombinant
protein bioreactor harvest material

Bioreactor harvest material components

| | Recombinant | | |
|---|---|---|---|
| Clarified | protein | DNA | HCP |
| Protein | (mg/mL) | (pg/mL) | (ng/mL) |
| Fc Fusion Protein | 1.65 | 72180600 | 94364 |
| Bispecific mAb | 3.40 | 202617600 | 915413 |
| mAb | 3.14 | 142781780 | 969014 |
| Concentrated mAb | 10.93 | 431469600 | 3314549 |

Effect of Concentrated Bioreactor Harvest Material on Inactivation of XMuLV with SIMULSOL™ SL 11W

The effect of SIMULSOL™ SL 11W on viral inactivation in concentrated clarified mAb harvest material is tested. Clarified mAb bioreactor harvest material containing the mAb and other impurities is concentrated about four-fold, through a 5 KDa molecular weight cutoff tangential flow filtration (TFF) process. The concentrated material containing the mAb and impurities is then immediately subjected to sterile filtration. The SIMULSOL™ SL 11W is then added to the clarified concentrated filtered material at a final concentration of 0.1% w/w (3 mM). The SIMULSOL™ 11 W containing material is then incubated at 3 different temperatures of 4° C., 18° C. and 30° C. Viral inactivation at the different temperatures is measured at time points ranging from 0 minutes to 180 minutes. The results as demonstrated in Table 17, show complete inactivation of XMuLV at 180 minutes at all 3 temperatures tested, even though the inactivation kinetics decreased at the lower temperatures when compared with the 30° C. sample. No effects on inactivation kinetics of the concentrated bioreactor material when compared to the unconcentrated bioreactor harvest material was observed (data not shown). This suggests that increased impurity levels do not affect viral inactivation by SIMULSOL™ SL 11W.

TABLE 17
Virus inactivation by 3 mM (0.1%) SIMULSOL ™ SL 11W in 4-
fold concentrated clarified mAb bioreactor harvest material

| Time point | LRF Achieved | | |
|---|---|---|---|
| (min) | 4° C. | 18° C. | 30° C. |
| 0 | 2.25 | 2.50 | 2.25 |
| 10 | 2.00 | 5.13 | 5.36 |
| 30 | 3.28 | 5.73 | ≥5.05 |
| 60 | 5.06 | ≥5.42 | ≥5.05 |
| 120 | ≥5.05 | ≥5.42 | ≥5.05 |
| 180 | ≥5.51 | ≥5.88 | ≥5.51 |

### Example 2. Determination of Removal of SIMULSOL™ SL 11W in Downstream

### Recombinant Protein Purification

### Undecyl Glycoside Quantification by Liquid Chromatography Mass Spectrometry (LC-MS)—Method A

LC-MS quantification is conducted on clarified mAb bioreactor harvest samples containing SIMULSOL™ SL 11W at concentrations ranging from 0.03 mM to 3 mM.

One microliter of the incubated sample is injected into a Waters Acquity® UPLC coupled to Waters SYNAPT® G2-Si mass spectrometer. For the quantitation, separations are performed on a Varian PLRP-S reversed-phase column (1×50 mm, 300 Å, 5 μm) at 80° C. using 0.05% trifluoroacetic acid (TFA) in H₂O as mobile phase A and 0.04% TFA in acetonitrile (ACN) as mobile phase B. The column is equilibrated with 15% mobile phase B for 1 minute and is linearly increased from 15% to 30% over a 3 minute timepoint, then is held at 30% B for 2 minutes and then held at 100% over 1 minute. After the column is held at 100% for 1 minute, it is re-equilibrated at 15% mobile phase B. SIMULSOL™ SL 11W is separated at 0.3 mL/min. between 1 to 6 min and is analyzed using an electrospray ionization (ESI) source. The mass spectrometer is operated at positive, sensitivity model, scan range of 100 to 1000 amu, capillary voltage of 3.2 kV, desolvation temperature of 450° C., desolvation gas flow of 900 L/hr, source temperature of 150° C. and a sample cone of 40 V. For characterization, the separation is conducted on Waters Acquity® UPLC BHE 300 Å C4 column (2.1×100 mm, 1.7 μm particle size) with the same mobile phases. Equilibrate the column at 20% mobile phase B for 1 minute, linearly increased from 20% to 25% over 14 minutes and then to 90% over 1 minute. After holding at 100% for 1.0 minute, re-equilibrate the column at 20% mobile phase B.

The extracted peak area of sodiated undecyl glycoside is measured. Under the LC-MS analysis conditions, a linear relationship was observed between extracted ion peak area and SIMULSOL™ SL 11W concentrations of 0.03 to 3 mM. When SIMULSOL™ SL 11W concentration was greater than 3 mM, the MS detector was saturated.

Undecyl Glycoside Quantification by Liquid Chromatography with tandem Mass Spectrometry (LC-MS/MS)—Method B

Standard solutions containing SIMULSOL™ SL 11W at concentrations ranging from 0.0008 mM to 0.012 mM are prepared in water or Protein A mainstream. After addition of SIMULSOL™ SL 11W, these samples were subjected to LC-MS/MS (MRM) analysis. Incubated sample (100 μL) is injected into the LC and split 1/20 post-column to MS. An Agilent 1260 Infinity II HPLC coupled to Sciex Triple Quad 5500 mass spectrometer is utilized for the analysis. For the quantitation, separations are performed on an Agilent Zorbax, 300SB-C8 reversed-phase column (4.6×50 mm, 300 Å, 3.5 μm) at 80° C. using 0.5 mM sodium acetate in water as mobile phase A and 0.5 mM sodium acetate in 95:5 acetonitrile:water as mobile phase B. The column is equilibrated at 20% mobile phase B for 2 minute, linearly increased from 20% to 90% over 10 minutes, held at 90% B for 2 minutes, the column is re-equilibrated at 20% mobile phase B. SIMULSOL™ SL 11 W is separated at 1.0 mL/min between 4.5 to 5.5 minutes and was analyzed using an electrospray ionization (ESI) source. Mass spectrometer is operated at positive, MRM mode with scanning transition ions (357.0 to 185.0), capillary voltage of 5.5 kV, desolvation temperature of 450° C., and desolvation gas flow of 55 L/hr.

Selected reactions monitoring (SRM/MRM (multiple reaction monitoring)), an LC-MS/MS method, is used to selectively detect the sodiated SIMULSOL™ SL 11W. SRM scans for a single precursor ion (mass of sodiated SIMULSOL™ SL 11W, 357.0 m/z) and after fragmentation, product ion (the most predominant fragment of sodiated SIMULSOL™ SL 11W, 185.0 m/z) is detected, and the peak area corresponding to this extracted transition ion is obtained.

Under the LC-MS/MS analysis conditions, the plots are linear between SIMULSOL™ SL 11W concentrations of 0.0008 mM to 0.012 mM in water and Protein A mainstream. These results suggest that LC-MS/MS is able to detect SIMULSOL™ SL 11W between 0.0008 mM to 0.012 mM and linearity of plots is independent of matrix. Therefore, this LC-MS/MS (SRM/MRM) method is sensitive with specificity for detection of SIMULSOL™ SL 11W and independent of the matrix the detergent is found in. The limit of detection (LOD) is 0.001 mM calculated from linearity.

### Protein a Chromatography and Detection of SIMULSOL™ SL 11W

Clarified mAb bioreactor harvest material solutions containing 0% (control), 0.3% and 1.0% w/w SIMULSOL™ SL 11W is subjected to MabSelect™ protein A chromatography. The Protein A chromatography is conducted on unfiltered clarified mAb harvest material containing SIMULSOL™ SL 11W. After the column is loaded, six column volumes of Tris/NaCl are used to wash the column prior to elution of the mAb with an acetic/citric acid buffer. The eluted mAb mainstream is collected and analyzed by LC-MS for the presence of SIMULSOL™ SL 11W.

The results of the analysis using LCMS (Method A) shows <0.03 mM of SIMULSOL™ SL 11W levels in the Protein A mainstream from the mAb bioreactor harvest material solutions containing 0.3% and 1.0% w/w SIMULSOL™ SL 11W. Using LC-MS/MS (Method B), the amount of SIMULSOL™ SL 11W is found to be 0.0005 mM in the Protein A mainstream from the mAb bioreactor harvest material solutions containing 0.3% w/w SIMULSOL™ SL 11W, which is lower than the LOD of 0.001 mM. These results indicate that Protein A chromatography is able to remove SIMULSOL™ SL 11W Simulsol SL 11W from the feedstream to a trace amount level, even without filtration steps prior to column loading.

### Example 3. Effect of SIMULSOL™ SL 11W on Recombinant Protein Quality

The effect of SIMULSOL™ SL 11W on the quality of recombinant proteins, is tested by comparing clarified mAb bioreactor harvest samples with and without 0.3% SIMULSOL™ SL 11W at 20° C. for 3 h, and then incubate at 4° C. for 16 h. The samples are first filtered through 0.2 μm glass fiber filters followed by 0.45 μm PVDF filters, and then subjected to Protein A purification. The samples are analyzed for the analytical properties as shown in Table 18 using techniques known in the art, such as capillary electrophoresis (CE), Size exclusion chromatography (SEC), isoelectric focusing (IEF) measure on an iCE™ instrument. The results as shown in Table 18, indicate that treatment with SIMULSOL™ SL 11W had no significant effect on any of the measured qualities as compared to no treatment with SIMULSOL™ SL 11W.

TABLE 18
mAb product quality analytical data with and
without SIMULSOL ™ SL 11W

| | No | 0.3% SIMULSOL ™ |
|---|---|---|
| Analytical Property | Detergent | SL 11W |
| CE reduced Heavy Chain (%) | 65.9 | 65.9 |
| CE reduced Light Chain (%) | 31.7 | 31.6 |
| CE non-reduced Main Peak (%) | 98.2 | 98.2 |
| SEC monomer (%) | 98.5 | 98.5 |
| SEC Aggregate (%) | 1.5 | 1.5 |
| IEF Main Peak (%) | 75.5 | 74.8 |
| IEF Total Acidic Variants (%) | 23.0 | 23.5 |
| IEF Total Basic Variants (%) | 1.5 | 1.6 |
| Chinese Hamster Ovary (CHO) | 336 | 283 |
| host cell protein (HCP) (ppm) | | |
| Residual Protein A (ppm) | 13.3 | 7.3 |
| DNA (ppb) | 21527 | 21473 |

1. A method of inactivating a virus in a feedstream comprising, adding to the feedstream an environmentally compatible detergent at a final concentration of about 0.05% w/w to about 1% w/w, wherein the environmentally compatible detergent comprises greater than about 40% undecyl alkyl glycoside.

2. The method of claim 1, wherein the detergent comprises about 40% to about 60% undecyl alkyl glycoside.

3. The method of 2, wherein the detergent comprises about 53% to about 57% undecyl alkyl glycoside.

4. The method of claim 1, wherein the detergent comprises greater than about 50% undecyl alkyl glycoside.

5. The method of claim 4, wherein the detergent comprises less than about 10% decyl alkyl glycoside.

6. The method of claim 5, wherein the detergent is an undecyl alkyl glycoside.

7. The method of claim 5, wherein the detergent is SIMULSOL™ SL 11W.

8. The method of claim 7, wherein the detergent is added to the feedstream at a concentration of about 0.1% w/w to about 1% w/w.

9. The method of claim 8, wherein the detergent is added to the feedstream at a final concentration of about 0.3% w/w.

10. The method of claim 9, wherein during said step of adding the detergent, the feedstream is at a temperature of about 15° C. to about 30° C.

11. The method of claim 10, wherein during said step of adding the detergent, the feedstream is at a temperature of about 15° C. to about 20° C.

12. The method of claim 11, wherein during said step of adding the detergent, the feedstream is at a pH of about 5.5 to about 8.0.

13. The method of claim 12, further comprising the step of incubating the detergent in the feedstream for about 1 minute to about 180 minutes.

14. The method of claim 13, wherein following said step of incubating, the detergent has a viral log reduction factor (LRF) in the feedstream of greater than about one, or greater than about two.

15. The method of claim 6, wherein the virus is a retrovirus, a herpesvirus, a flavivirus, a poxvirus, a hepadnavirus, a hepatitis virus, an orthomyxovirus, a paramyxovirus, a rhabdovirus, or a togavirus.

16. The method of claim 14, wherein the virus is a retrovirus, a herpesvirus, a flavivirus, a poxvirus, a hepadnavirus, a hepatitis virus, an orthomyxovirus, a paramyxovirus, a rhabdovirus, or a togavirus.

17. The method of claim 1, further comprising the step of incubating the feedstream and the detergent for about 60 minutes to about 180 minutes at a temperature of about 4° C. to about 30° C. and at a pH of about 5.5 to about 8.0, wherein the detergent is added to the feedstream at a final concentration of about 0.3% w/w, and wherein the detergent in the feedstream has a viral log reduction factor (LRF) of greater than about 5.

18. The method of claim 17, wherein the feedstream is a harvested cell culture fluid, a capture pool or a recovered product pool.

19. The method of claim 18, wherein the capture poor or the recovered product pool is a protein A pool, a protein G pool or a protein L pool affinity chromatography pool.

20. The method of claim 18, wherein the capture pool or recovered product pool is a mixed mode chromatography pool.

21. The method of claim 19, further comprising the step of filtering the feedstream containing the detergent, wherein the feedstream is subjected to at least one filtration step.

22. The method of claim 21, wherein the feedstream is subjected to at least a second filtration step.

23. The method of claim 21, further comprising the step of subjecting the filtered feedstream to an affinity chromatography column.

24. The method of claim 23, wherein the affinity chromatography column is a Protein A column.

25. The method of claim 24, wherein the feedstream containing the detergent is subjected to affinity chromatography.

26. The method of claim 25, wherein the feedstream comprises a protein, wherein the protein is an antibody, an Fc fusion protein, an immunoadhesin, an enzyme, a growth factor, a receptor, a hormone, a regulatory factor, a cytokine, an antigen, a peptide, or a binding agent.

27. The method of claim 26, wherein the antibody is a monoclonal antibody, a chimeric antibody, a humanized antibody, a human antibody, a bispecific antibody, or an antibody fragment produced in a mammalian cell.

28. The method of 27, wherein the step of adding the detergent to the feedstream does not significantly alter turbidity of the feedstream-and the step of adding the detergent to the feedstream does not alter final product quality of the purified feedstream protein.

29. The method of claim 28, wherein the detergent contains a stabilizing agent.

30. The method of claim 29, wherein the stabilizing agent is monopropylene glycol.

31. A method of inactivating a virus in a feedstream comprising the steps of:
adding to the feedstream SIMULSOL™ SL 11W at a concentration of about 0.1% w/w to about 0.3% w/w;
incubating the SIMULSOL™ SL 11W in the feedstream for about 180 minutes, wherein the viral log reduction factor (LRF) of SIMULSOL™ SL 11W in the feedstream is greater than about 5;
filtering said feedstream containing the SIMULSOL™ SL 11W; and
subjecting said filtered feedstream to a Protein A affinity chromatography column; wherein, said step of adding and incubating the feedstream at a temperature of about 4° C. to about 30° C. and at a pH of about 5.5 to about 8.0.
